(19) United States
(12) Patent Application Publication
(10) Pub. No.: US 2025/0049924 A1
(43) Pub. Date: Feb. 13, 2025

(54) Abuse-Deterrent Pharmaceutical Composition with Aversion Properties

(71) Applicant: AARDVARK THERAPEUTICS INC., San Diego, CA (US)

(72) Inventors: Jane Wu LEE, San Diego, CA (US); Tien-Li LEE, San Diego, CA (US)

(73) Assignee: AARDVARK THERAPEUTICS INC., San Diego, CA (US)

(21) Appl. No.: 18/924,880

(22) Filed: Oct. 23, 2024

Related U.S. Application Data
(63) Continuation of application No. PCT/US23/20279, filed on Apr. 27, 2023.
(60) Provisional application No. 63/335,701, filed on Apr. 27, 2022.

(51) Int. Cl. A61K 47/18; A61K 9/00; A61K 9/16; A61K 9/48; A61K 45/06

(52) U.S. Cl. CPC A61K 47/186; A61K 9/0053; A61K 9/1611; A61K 9/4858; A61K 9/4866; A61K 45/06

(57) ABSTRACT

There is disclosed an improved abuse-deterrent pharmaceutical composition comprising a controlled pharmaceutical substance in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts (including denatonium acetate (DA), denatonium benzoate (DB), denatonium chloride, denatonium citrate, denatonium maleate, denatonium saccharide, and denatonium tartrate) chlorpheniramine, diphenidol, famotidine, haloperidol, quinine, parthenolide, and aristolochic acid. Preferably, the controlled pharmaceutical substance is an amphetamine or a pharmaceutically acceptable salt thereof. Preferably, the bitter receptor agonist is a denatonium salt selected from DA, DB, denatonium chloride, denatonium citrate, denatonium maleate, denatonium saccharide, and denatonium tartrate provided at from about 0.5 mg per dose administered to about 10 mg per dose administered. There is further disclosed a use for denatonium acetate (DA) as a pharmaceutical grade aversive agent to be added to abuse-deterrent formulations to deter snorting or smoking a controlled pharmaceutical substance at a dose of about 0.5 mg to about 10 mg per dose.

### CROSS-REFERENCE TO RELATED APPLICATIONS

This application claims benefit to U.S. Provisional Application No. 63/335,701, filed 27 Apr. 2022, which is incorporated by reference herein.

### TECHNICAL FIELD

The present disclosure provides an improved abuse-deterrent pharmaceutical composition comprising a controlled pharmaceutical substance in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts (including denatonium acetate (DA), denatonium benzoate (DB), denatonium chloride, denatonium citrate, denatonium maleate, denatonium saccharide, and denatonium tartrate) chlorpheniramine, diphenidol, famotidine, haloperidol, quinine, parthenolide, and aristolochic acid. Preferably, the controlled pharmaceutical substance is an amphetamine or a pharmaceutically acceptable salt thereof. Preferably, the bitter receptor agonist is a denatonium salt selected from DA, DB, denatonium chloride, denatonium citrate, denatonium maleate, denatonium saccharide, and denatonium tartrate provided at from about 0.5 mg per dose administered to about 10 mg per dose administered. The present disclosure further provides a use for denatonium acetate (DA) as a pharmaceutical grade aversive agent to be added to abuse-deterrent formulations to deter snorting or smoking a controlled pharmaceutical substance.

### BACKGROUND

Current abuse deterrent formulations involve limiting the potential for abuse by limiting the ability to manipulate a therapeutic formulation, particularly extended release formulations to achieve an abused bolus dose. One such abuse-deterrent formulation (“Vallon formulation”) of a controlled substance that would be provided in a capsule for prescribed administration, such as for treatment of ADHD (attention deficit hyperactivity disorder), has been tested in clinical trials that is described in U.S. Pat. No. 9,931,303, the disclosure of which is incorporated by reference herein. The Vallon formulation was able to deter iv administration/abuse due to its waxy excipients made it too difficult to push through a syringe. However, the Vallon formulation failed to deter abuse from snorting the contents of its capsule dosage form. Moreover, the Vallon formulation was not even evaluated as to whether or not it could deter smoking the contents of its formulation added to a capsule dosage form.

There are no pharmaceutical grade aversive agents. There is denatonium benzoate (DB) commercially available for consumer products, such as used on laundry detergent pods (Tide Pods) for purposes of deterring ingestion by children. However, no aversive agents have been used to deter self-medication of controlled substances by addicted individuals. Moreover, denatonium salts, like commercial DB, tends to adhere to surfaces and does not flow well as a powdery substance, making it difficult to manufacture under GMP (good manufacturing practices) conditions needed for a pharmaceutical product.

Therefore, there is a need in the art to be able to deter abuse of controlled substance formulations seeking bolus systemic exposure of the controlled substance by bolus administration by iv injection, sporting and smoking routes of abuse administration.

### SUMMARY

The present disclosure provides an improved abuse-deterrent pharmaceutical composition comprising a controlled pharmaceutical substance, selected from the group consisting of an amphetamine (including dextroamphetamine, or a pharmaceutically acceptable salt thereof), methylphenidate, barbiturates (including amobarbital, secobarbital, butalbital/acetaminophen or aspirin/caffeine), an opioid (including hydrocodone, oxycodone, oxymorphone, morphine, codeine and fentanyl), and a benzodiazepine (including alprazolam, chlordiazepoxide, clonazepam, clorazepate, diazepam, estazolam, flurazepam, and lorazepam), and sleep medications (including zolpidem, zaleplon, and eszopiclone) in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts (including denatonium acetate (DA), denatonium benzoate (DB), denatonium chloride, denatonium citrate, denatonium maleate, denatonium saccharide, and denatonium tartrate) chlorpheniramine, diphenidol, famotidine, haloperidol, quinine, parthenopid, and aristolochic acid. Preferably, the bitter receptor agonist is a denatonium salt selected from the group consisting of DA, DB, denatonium chloride, denatonium citrate, denatonium maleate, denatonium saccharide, and denatonium tartrate, even more preferably DA. Preferably, the denatonium salt is formulated into a granule comprising talc to allow for better flow of granules to be added to a gooey and waxy excipient base. Denatonium salts retain their bitter taste characteristics if combusted (oxidized).

The present disclosure provides an improved abuse-deterrent pharmaceutical composition comprising a controlled pharmaceutical substance in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts (including denatonium acetate (DA), denatonium benzoate (DB), denatonium chloride, denatonium citrate, denatonium maleate, denatonium saccharide, and denatonium tartrate) chlorpheniramine, diphenidol, famotidine, haloperidol, quinine, parthenolide, and aristolochic acid. Preferably, the controlled pharmaceutical substance is an amphetamine or a pharmaceutically acceptable salt thereof. Preferably, the bitter receptor agonist is a denatonium salt selected from DA, DB, denatonium chloride, denatonium citrate, denatonium maleate, denatonium saccharide, and denatonium tartrate provided at from about 0.5 mg per dose administered to about 10 mg per dose administered. The present disclosure further provides a use for denatonium acetate (DA) as a pharmaceutical grade aversive agent to be added to abuse-deterrent formulations to deter snorting or smoking a controlled pharmaceutical substance.

The disclosure provides an abuse-deterrent and aversive formulation having a controlled pharmaceutical substance; at least two waxy-gel forming excipients, and an aversive agent that is a TAS2R bitter agonist. The controlled pharmaceutical substance, may target the central nervous system and/or may be used to treat psychiatric disorders or treating pain. A preferred controlled pharmaceutical substance is an amphetamine such as dextroamphetamine, or a pharmaceutically acceptable salt thereof. Specifically, the controlled pharmaceutical substance has a formula:

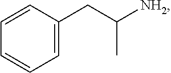

or a pharmaceutically acceptable salt thereof. In further specific embodiments, the controlled pharmaceutical substance is the S enantiomer, or a pharmaceutically acceptable salt thereof. Excipients include for example. PEG ester, poloxamer, water-soluble anionic polysaccharide, and carboxymethylcellulose. Preferably, the bitter receptor agonist is a denatonium salt selected from DA, DB, denatonium chloride, denatonium citrate, denatonium maleate, denatonium saccharide, and denatonium tartrate, even more preferably DA. Preferably, the amount of DA in a capsule is from about 1 mg to about 30 mg. More preferably, the amount of DA in each capsule is from about 2 mg to about 18 mg. Most preferably, a sufficient aversive dose level of DA is approximately 10 mg. Preferably, the abuse deterrent formulation is in the form of a capsule.

In some embodiments, the abuse-deterrent formulation comprises a controlled pharmaceutical substance, PEG ester, poloxamer, water-soluble anionic polysaccharide, and an aversive agent. In specific embodiments, the PEG ester is polyoxyl stearate; the poloxamer is poloxamer 124; the water-soluble anionic polysaccharide is gellan gum, and the aversive agent is a denatonium salt selected from the group consisting of DA, DB, denatonium chloride, denatonium citrate, denatonium saccharide, denatonium maleate, and denatonium tartrate. In some embodiments, the ratio of poloxamer:polysaccharide:PEG ester is about 40:30:30.

In some embodiments, the PEG ester is polyoxyl stearate; the water-soluble anionic polysaccharide is gellan gum, and the aversive agent is a denatonium salt selected from the group consisting of DA, DB, denatonium chloride, denatonium citrate, denatonium saccharide, denatonium maleate, and denatonium tartrate. In further specific embodiments, the ratio of PEG ester:water-soluble anionic polysaccharide is about 70:30. Preferably, the bitter receptor agonist is a denatonium salt selected from DA, DB, denatonium chloride, denatonium citrate, denatonium maleate, denatonium saccharide, and denatonium tartrate, even more preferably DA. Preferably, the amount of DA in a capsule is from about 1 mg to about 30 mg. More preferably, the amount of DA in each capsule is from about 2 mg to about 18 mg. Most preferably, a sufficient aversive dose level of DA is approximately 10 mg. Preferably, the poloxamer is poloxamer 124. Preferably, the water-soluble anionic polysaccharide id gellan gum. Preferably the PEG ester is polyoxyl stearate. Preferably the ratio of poloxamer:water soluble anionic polysaccharide:PEG ester is about 40:30:30.

In yet another embodiment, the abuse-deterrent formulation comprises controlled pharmaceutical substance, PEG ester, and carboxymethylcellulose. In specific embodiments, the PEG ester is polyoxyl stearate. In further specific embodiments, the ratio of PEG ester and carboxymethylcellulose is about 70:30. Preferably, the bitter receptor agonist is a denatonium salt selected from DA, DB, denatonium chloride, denatonium citrate, denatonium maleate, denatonium saccharide, and denatonium tartrate, even more preferably DA. Specifically, the disclosure provides, an abuse-deterrent formulation, including a controlled pharmaceutical substance, a poloxamer, a water-soluble anionic polysaccharide, and a PEG ester. The controlled pharmaceutical substance is

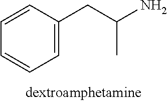

or a pharmaceutically acceptable salt thereof. Alternatively, is the S enantiomer of amphetamine, or a pharmaceutically acceptable salt thereof. Preferably, the controlled pharmaceutical substance is dextroamphetamine, or a pharmaceutically acceptable salt thereof. The pharmaceutically acceptable salt is for example, a sulfate salt. The unit dose of the controlled pharmaceutical substance in the formulation is from about 10 mg to about 50 mg. Preferably, the abuse deterrent formulation is in the form of a capsule. The capsule is for example gelatin.

The poloxamer is poloxamer 124. The water-soluble anionic polysaccharide is gellan gum. The PEG ester is polyoxyl stearate. The ratio of poloxamer:water-soluble anionic polysaccharide:PEG ester is about. 40:30:30.

The abuse-deterrent formulation included 33-43 wt % of poloxamer; 24-32 wt % of water-soluble anionic polysaccharide; and 24-32 wt % of PEG ester. The ratio of poloxamer 124:gellan gum:polyoxyl stearate is about 40:30:30. The poloxamer is Kollisoly P124, the water-soluble anionic polysaccharide is Kelcogel CGHA, and the PEG ester is Gelucire 48/16.

A preferred formulation includes:

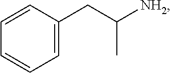

or the S enantiomer (dextroamphetamine), or a pharmaceutically acceptable salt as thereof as the controlled pharmaceutical substance, poloxamer 124, gellan gum, and polyoxyl stearate where the ratio of poloxamer 124:gellan gum:polyoxyl stearate is about 40:30:30. In some embodiments, the poloxamer 124 is Kollisoly P124, the gellan gum is Kelcogel CGHA, and the polyoxyl stearate is Gelucire 48/16. Preferably, the bitter receptor agonist is a denatonium salt selected from DA, DB, denatonium chloride, denatonium citrate, denatonium maleate, denatonium saccharide, and denatonium tartrate, even more preferably DA.

### DETAILED DESCRIPTION

The present disclosure provides an improved abuse-deterrent pharmaceutical composition comprising a controlled pharmaceutical substance, selected from the group consisting of an amphetamine (including dextroamphetamine, or a pharmaceutically acceptable salt thereof), metylphenidate, barbiturates (including amobarbital, secobarbital, butalbital/acetomenophen or aspirin/caffeine), an opioid (including dydrocodone, oxycodone, oxymorphone, morphine, codeine and fentanyl), and a benzodiazepine (including alprazolam, chlordiazepoxide, clonazepam, clorazepate, diazepam, estazolam, flurazepam, and lorazepam), and sleep medications (including zolpidem, zaleplon, and eszopiclone) in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts (including denatonium acetate (DA), denatonium benzoate (DB), denatonium citrate, denatonium maleate, denatonium saccharide, and denatonium tartrate) chlorpheniramine, diphenidol, famotidine, haloperidol, quinine, parthenolide, and aristolochic acid. Preferably, the bitter receptor agonist is a denatonium salt selected from DA, DB, denatonium chloride, denatonium citrate, denatonium maleate, denatonium saccharide, and denatonium tartrate, even more preferably DA. Denatonium salts retain their bitter taste characteristics if combusted (oxidized). Preferably, the controlled substance is dextroamphetamine that is water-soluble and the aversive agent is DA, which is also water-soluble.

Without being bound by theory, and based upon the experience provided in the Example herein, it is thought that both the water soluble amphetamine active controlled-release substance, and the aversive component water-soluble DA, solubilize within the nasal mucosa passages and then get to the back of the mouth and back of tongue upon snorting or within about 10 minutes from snorting. DA, according to completed clinical trials and preclinical studies, tends to remain in the GI tract upon oral administration and have less than 1% of the dose administered found systemically. Accordingly, it is likely that upon exposure to the nasal mucosa, it will also remain within the nasal lumen and flow with the nasal lumen toward the back of the mouth and eventually down the GI tract, rather than become absorbed systemically. This is in contrast to a controlled substance, such as an amphetamine, which is known to provide a bolus dose to the brain to provider a “high” experience for the drug abuser. This allows a drug abuser, snorting the disclosed formulation to experience a bolus dose of the controlled substance for a “high” while simultaneously, or shortly thereafter, experience a disgusting, revolting, vomit-inducing, sickening (but not life-threatening) massively unpleasant experience from the aversive agent, such as DA. The result, similar to that was seen with rat toxicology testing by gavage administration, was an avoidance, at all costs, from the disclosed formulation due to the extreme unpleasantness/disgust, etc. experienced after the first administration.

The use of the term “about” includes and describes the value or parameter per se. For example, “about x” includes and describes “x” per se. In some embodiments, the term “about” when used in association with a measurement, or used to modify a value, a unit, a constant, or a range of values, refers to variations of +/−5%, or +/−10%.

Misuse or abuse of a controlled substance is defined as (1) administration or dosing differing from the prescribed methods, (2) taking medications not prescribed to the individual, or (3) taking medications for the side effects and not for the prescribed issue. Moreover, abuse deterrent formulation (ADF) is defined as “: Medications with the goals being reduction in drug-liking qualities, limiting the bioavailability, preventing dosage through alternative routes, or making abuse of the reformulation less rewarding.” (Green, Pain Management 2010; 1-3. 2.; and Cicero and Surratt, N. Engl. J. Med. 2012; 367:187-189).

### Excipients

The excipients of the present abuse deterrent pharmaceutical composition are designed for contents of the capsule to be in a waxy gel-like state with high viscosity to physically deter the ability to inject the capsule contents. However, such waxy contents were unsuccessful deterring the formulation having just excipients from snorting the capsule contents as a preferred means for abusing the pharmaceutical to achieve a bolus administration of the controlled pharmaceutical substance. Accordingly, the present disclosure further comprises an aversive agent to achieve complete abuse deterrence.

The formulation contains one or more excipients. The excipients are selected to prevent abuse of the controlled pharmaceutical substance.

Suitable abuse deterrent excipients may display one or more of the following properties, high melting point excipients resistant to heating and that prevent injecting; taste modifiers which prevent covert administration, snorting and dose dumping; water insolubles that are resistant to extraction and that prevent drink adulteration; waxy excipients that prevent snorting; viscosity modifiers resistant to dissolution and that prevent injecting and dose dumping; low density excipients that prevent drink adulteration; and dyes that disclose abuse of the pharmaceutical controlled pharmaceutical substance.

Exemplary excipients include for example thermosoftening pharmaceutical bases including waxes, poloxamers, macrogol glycerides, PEGs, glycerol monooleates or monostearates, PEG esters such as polyoxyl stearate, hydrogenated or partially hydrogenated glycerides and hard fats such as beeswax, poloxamer 188, poloxamer 124, Gelucires™ polyethylene 6000, glycerol monostearate, hydrogenated palm kernel oil, hydrogenated cottonseed oil, Softisan™ 138, Gelucire™ 40/01, hexadecan-1-ol; Thixotropes such as fumed silica and pulverised attapulgite and viscosity modifiers such as hydroxyl propyl methyl cellulose or Gellan gum to increase viscosity or the standard pharmaceutical or food grade oils such as fractionated coconut oil, soybean oil etc. to decrease viscosity.

Preferably, the abuse deterrent excipients include a poloxamer, a water-soluble anionic polysaccharide and a PEG ester. Preferably, the poloxamer is poloxamer 124 such as Kollisolv™. Preferably, the water soluble anionic polysaccharide is gellan gum such as Kecogel CGHA™. Preferably, the PEG ester is polyoxyl stearate such as Gelucire 48/16™.

The abuse deterrent pharmaceutical composition of a controlled substance may be in a capsule form, such as a hard shell liquid filled capsule. For example, the capsule comprises gelatin. Alternatively, the capsule comprises hydroxypropyl methylcellulose (HPMC), pullalan or other hard shell material.

The abuse-deterrent formulation of the controlled substance comprises at least two excipients selected from PEG ester, poloxamer, water-soluble anionic polysaccharide, and carboxymethylcellulose. The abuse deterrent formulation comprises a bitter receptor agonist to act as an aversive for abuse attempts of snorting or smoking the contents of the formulation. A the bitter receptor agonist is a denatonium salt selected from DA, DB, denatonium chloride, denatonium citrate, denatonium maleate, denatonium saccharide, and denatonium tartrate, even more preferably DA. In some embodiments, the abuse-deterrent formulation of the controlled substance comprises at least two excipients selected from Kollisolv™ P124, Kolliphor™ EL, Kolliphor™ RH40, Tween™ 20, Gelucire™ 48/16, Gelucire™ 44/14, Super refined Corn Oil, Aerosil™ 200, Luxura™, Xantural™ 75, Kelcogel™ CGHA, CMC 7H3SF, Methocel™ A4CP, Gelatin Type B 220 Bloom, and PEG6000. In some embodiments, the abuse-deterrent formulation of dextroamphetamine comprises a controlled pharmaceutical substance, PEG ester, poloxamer, and water-soluble anionic polysaccharide. In specific embodiments, the PEG ester is polyoxyl stearate; the poloxamer is poloxamer 124; and the water-soluble anionic polysaccharide is gellan gum. In some embodiments, the ratio of poloxamer:polysaccharide:PEG ester is about 40:30:30.

In some embodiments, the abuse-deterrent formulation of dextroamphetamine comprises controlled pharmaceutical substance, PEG ester, and water-soluble anionic polysaccharide. In specific embodiments, the PEG ester is polyoxyl stearate; and the water-soluble anionic polysaccharide is gellan gum. In further specific embodiments, the ratio of PEG ester:water-soluble anionic polysaccharide is about 70:30. The abuse deterrent formulation further comprises a bitter receptor agonist to act as an aversive for abuse attempts of snorting or smoking the contents of the formulation. A bitter receptor agonist is a denatonium salt selected from DA, DB, denatonium chloride, denatonium citrate, denatonium maleate, denatonium saccharide, and denatonium tartrate, even more preferably DA.

In yet another embodiment, the abuse-deterrent formulation comprises controlled pharmaceutical substance, PEG ester, and carboxymethylcellulose. In specific embodiments, the PEG ester is polyoxyl stearate. The abuse deterrent formulation further comprises a bitter receptor agonist to act as an aversive for abuse attempts of snorting or smoking the contents of the formulation. A bitter receptor agonist is a denatonium salt selected from DA, DB, denatonium citrate, denatonium maleate, denatonium saccharide, and denatonium tartrate, even more preferably DA. In further specific embodiments, the ratio of PEG ester and carboxymethylcellulose is about 70:30.

In some embodiments, the abuse-deterrent formulation comprises a controlled pharmaceutical substance, Kollisolv™ P124, Kelcogel™ CGHA, and Gelucire™ 48/16. In further specific embodiments, the ratio of Kollisolv™ P124, Kelcogel™ CGHA, and Gelucire™ 48/16 is about 40:30:30.

In some embodiments, the abuse-deterrent formulation comprises a controlled pharmaceutical substance, Gelucire™ 48/16 and Kelcogel™ CGHA. In further specific embodiments, the ratio of Gelucire™ 48/16 and Kelcogel™ CGHA is about 70:30. The abuse deterrent formulation further comprises a bitter receptor agonist to act as an aversive for abuse attempts of snorting or smoking the contents of the formulation. A bitter receptor agonist is a denatonium salt selected from DA, DB, denatonium chloride, denatonium citrate, denatonium maleate, denatonium saccharide, and denatonium tartrate, even more preferably DA.

In some embodiments, the abuse-deterrent formulation comprises a controlled pharmaceutical substance, Kolliphor™ EL and CMC 7H3SF. In further specific embodiments, the ratio of Kolliphor™ EL and CMC 7H3SF is about 70:30.

In some embodiments, the abuse-deterrent formulation comprises one or more controlled pharmaceutical substances is dextroamphetamine saccharate or dextroamphetamine sulfate. In preferred embodiments the abuse-deterrent formulation of dextroamphetamine, a poloxamer, a water-soluble anionic polysaccharide, a PEG ester and a bitter receptor agonist to act as an aversive for abuse attempts of snorting or smoking the contents of the formulation.

The poloxamer is poloxamer 124. The water-soluble anionic polysaccharide is gellan gum. The PEG ester is polyoxyl stearate. The ratio of poloxamer:water-soluble anionic polysaccharide:PEG ester is about 40:30:30. The abuse-deterrent formulation included 33-43 wt % of poloxamer; 24-32 wt % of water-soluble anionic polysaccharide; and 24-32 wt % of PEG ester. The ratio of poloxamer 124:gellan gum:polyoxyl stearate is about 40:30:30. The poloxamer is Kollisolv™ P124, the water-soluble anionic polysaccharide is Kelcogel™ CGHA, and the PEG ester is Gelucire™ 48/16.

A preferred formulation includes dextroamphetamine, or a pharmaceutically acceptable salt as thereof poloxamer 124, gellan gum, and polyoxyl stearate where the ratio of poloxamer 124:gellan gum:polyoxyl stearate is about 40:30:30. In some embodiments, the poloxamer 124 is Kollisolv™ P124, the gellan gum is Kelcogel™ CGHA, and the polyoxyl stearate is Gelucire™ 48/16.

### Controlled Pharmaceutical Substance

Controlled pharmaceutical substances that have been abused are selected from the group consisting of amphetamine, dextroamphetamine, metylphenidate, amobarbital, secobarbital, butalbital/acetomenophen or aspirin/caffeine, dydrocodone, oxycodone, oxymorphone, morphine, codeine, fentanyl, alprazolam, chlordiazepoxide, clonazepam, clorazepate, diazepam, estazolam, flurazepam, lorazepam, zolpidem, zaleplon, and eszopiclone.

“Amphetamine” has the formula:

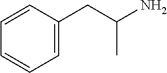

“Dextroamphetamine” is the S enantiomer of amphetamine and has the formula:

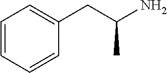

A unit dose of dextroamphetamine or amphetamine is between about 10-50 mg. For example, the unit dose is 5 mg, 10 mg, 15 mg, 20 mg, 25 mg, 30 mg, 35 mg, 40 mg, 45 mg, or 50 mg. The unit does is administered once, twice, three, or four times daily. The daily does is between 5 mg and 100 mg. For example the daily dose is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95, mg or 100 mg.

### Bitter Aversive Compounds

A bitter-agonist compound selected from the group consisting of denatonium salts (including denatonium acetate (DA), denatonium benzoate (DB), denatonium citrate, denatonium maleate, denatonium saccharide, and denatonium tartrate) chlorpheniramine, diphenidol, famotidine, haloperidol, quinine, parthenolide, and aristolochic acid. Preferably, the bitter receptor agonist is a denatonium salt selected from DA, DB, denatonium chloride, denatonium citrate, denatonium maleate, denatonium saccharide, and denatonium tartrate, even more preferably DA. Denatonium salts retain their bitter taste characteristics if combusted (oxidized).

DB is commercially available but has not been developed for pharmaceutical use. However, DA has been used as an active pharmaceutical agent as a bitter receptor agonist, not as an aversive, in formulations that avoid oral cavity exposure. The pharmacokinetics of DA show that it is substantially gut restricted after oral exposure, allowing for clinical use based on both toxicology and pharmacokinetic data. Moreover, any systemic administration (i.e., intravenous administration) showed a high NOAEL (no adverse effect level) to allow for low dose use in abuse deterrent formulations as an aversive to strongly deter snorting or smoking forms of abuse.

Denatonium acetate (DA) is preferably a monohydrate salt having the formula:

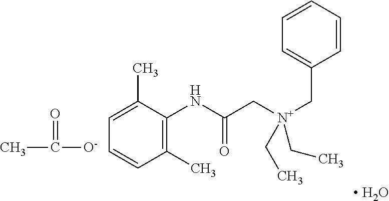

or a hydrate thereof. Preferably, the salt is a crystalline monohydrate.

A process for synthesizing DA begins with Lidocaine base to make Denatonium Hydroxide and then DA anhydropus.

Step 1: Synthesis of Denatonium Hydroxide from Lidocaine

To a reflux apparatus add 25 g of lidocaine, 60 ml of water, and 17.5 g of benzyl chloride with stirring and heating in 70-90° C. The solution needs to be heated and stirred in the before given value for 24 h, the solution needs to be cooled down to 30° C. The unreacted reagents are removed with 3×10 mL of toluene. With stirring dissolve 65 g of sodium hydroxide into 65 mL of cold water and add it to the aqueous solution with stirring over the course of 3 h. Filter the mixture, wash with some water and dry in open air. Recrystallize in hot chloroform or hot ethanol.

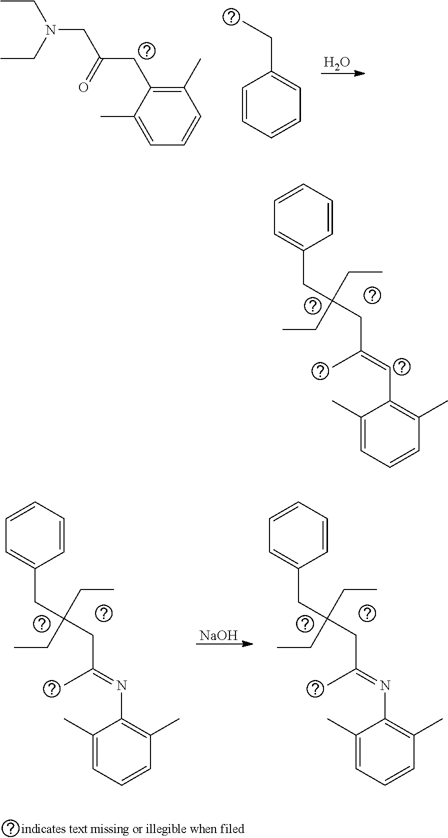

Step 2: Preparation of Denatonium Acetate Anhydride from Denatonium Hydroxide.

To a reflux apparatus 10 g of denatonium hydroxide (MW: 342.475 g/mol, 0.029 mol), 20 mL of acetone, and 2 g of acetic acid glacial (0.033 mol) dissolved in 15 mL of 5 acetone is added, the mixture is stirred and heated to 35° C. for 3 h. Then evaporated to dryness and recrystallized in hot acetone.

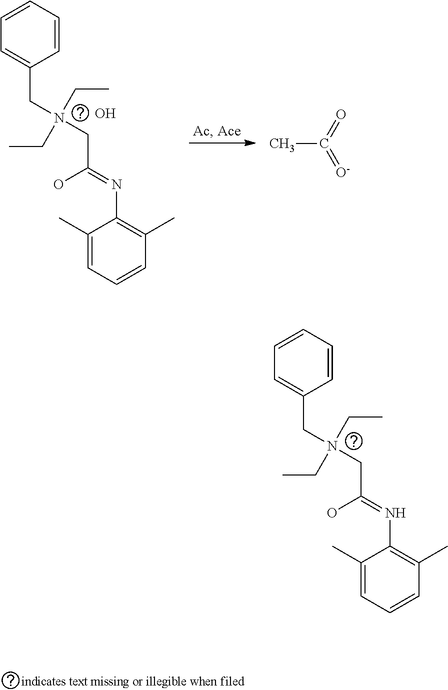

For Step 3, DA anhydrous was maintained in an organic layer and was distilled under vacuum until the temperature reached 65-67° C. at <150 torr. Methyl isobutyl ketone was added and refluxed under vacuum to remove water via azeotropic distillation to form DA monohydrate. DA was crystalized by adding isopropyl alcohol. Residual salts were removed. The mixture was distilled under vacuum. Next, methyl isobutyl ketone was added and then water. The temperature was lowered to <10° C. The remaining solid was isolated and washed with methyl isobutyl ketone to produce the final DA (denatonium acetate monohydrate).

A denatonium salt is provided in a capsule or coated tablet formulation of 100 mg in an amount of from about 1 to about 200 micrograms of denatonium, preferably from about 5 to about 100 microgram-of denatonium and most preferably from about 20 to about 75 micrograms denatonium. In order to achieve aversion if an abuser were to snort or smoke an abuse deterrent formulation, optimally the concentration of denatonium in the formulation should achieve about 50 ppm (parts per million).

### Example

One brave co-inventor attempted to “snort” a formulation of DA in either a granule form having talc as an excipient as DA is provided in clinical trials (Aardvark Therapeutics, Inc phase 1 and 2 studies for obesity) for oral administration in a capsule designed to prevent DA material from exposure to bitter taste receptors in the oral cavity, including the tongue. In a first round of “dose escalation” of a saline having DA in granules starting around 0.02 mg of DA (wherein a granule weighs about 0.1 mg including the talc based excipients) in the volume of liquid snorted, it took a dose of around 0.1 mg DA (10 granules). The three attempts were 1 granule, 5 granules and lastly 10 granules. The report from the “volunteer was “For me, the effect wasn't immediately noticeable, but within 10 minutes, I noted an extremely bitter taste in the back of my throat and posterior portion of my tongue. This sensation peaked around 15-20 min, and lasted about an hour. Was certainly annoying and not pleasant. It did not elicit nausea or vomiting.”

And in some of the animal toxicology studies (particularly in rats), where DA was administered orally by gavage, some less skilled gavage technicians was encountering highly aversive and desperate behavior from rats, resulting in loss of control of desperate animals.

In view of the results with a granule formulation of DA, the day before filing this patent application, the co-inventor volunteer tried the snorting experiment again, but this time, using neat DA that was a powder dissolved in saline. Just one “snort” was attempted of 1 mg of DA in saline. It should be noted that the completed clinical trials of DA used a dose of 200 mg of DA taken orally in a capsule that dissolves in the stomach, avoiding mouth exposure. The report from our volunteer indicated: “Just snorted 1.0 mg of ARD-101 [DA] API [active pharmaceutical ingredient] . . . OMG.

So terrible. Not exactly sure if it's because it's a larger dose load than the 1 mg of granules snorted before, but the effect is significantly and measurably more disgusting. Like with the granules, there is no immediate aversive sensation. Maybe around minute 7-8 after snorting dose, the bitterness became apparent, with peak effect starting around minute 10-15. Sensation of extreme bitterness in the back of the throat and the posterior tongue. Drinking water almost makes it worse!

It's been over 30 min since the snorting and the bitterness has hardly diminished. If I ate breakfast, I might have vomited already. If this tracks with the earlier experience with the granules, the feeling may last for an hour or so, but in any case, think ARD-101 [DA] would indeed be a measurable deterrent to snorting.” (emphasis supplied).

A later text read: “It's been almost 6 hours and I still taste it. Bleh.”

Accordingly, the brave and tortured volunteer has demonstrated that a drug abuser may abuse by snorting the present formulation a first time, then experience what the volunteer experienced, and will likely never go through such a tortured experience again.

We claim:

1. An abuse-deterrent pharmaceutical composition comprising a controlled pharmaceutical substance selected from the group consisting of amphetamine, dextroamphetamine, metylphenidate, amobarbital, secobarbital, butalbital/acetomenophen or aspirin/caffeine, dydrocodone, oxycodone, oxymorphone, morphine, codeine, fentanyl, alprazolam, chlordiazepoxide, clonazepam, clorazepate, diazepam, estazolam, flurazepam, lorazepam, zolpidem, zaleplon, and eszopiclone, in a formulation comprising at least two gel-forming excipients selected from the group consisting of PEG ester, poloxamer, water-soluble anionic polysaccharide, and carboxymethylcellulose, and a bitter-agonist compound selected from the group consisting of denatonium salts, denatonium acetate (DA), denatonium benzoate (DB), denatonium chloride, denatonium citrate, denatonium maleate, denatonium saccharide, and denatonium tartrate, chlorpheniramine, diphenidol, famotidine, haloperidol, quinine, parthenolide, and aristolochic acid.

2. The abuse-deterrent pharmaceutical composition of claim 1, wherein the bitter receptor agonist is a denatonium salt selected from DA, DB, denatonium chloride, denatonium citrate, denatonium maleate, denatonium saccharide, and denatonium tartrate.

3. The abuse-deterrent pharmaceutical composition of claim 2, wherein the concentration of denatonium in the improved abuse-deterrent pharmaceutical composition is about 50 ppm.

4. The abuse-deterrent pharmaceutical composition of claim 3, wherein the denatonium salt is denatonium acetate monohydrate (DA) at a dose administered/snorted of from about 0.5 mg to about 10 mg of total DA.

5. The abuse-deterrent pharmaceutical composition of claim 4, wherein the (DA) at a dose administered/snorted of from about 0.5 mg to about 10 mg of total DA.

6. The abuse-deterrent and aversive formulation of claim 4, wherein the gel forming excipients are selected from the group consisting of PEG ester, poloxamer, water-soluble anionic polysaccharide, and carboxymethylcellulose.

7. The abuse-deterrent and aversive formulation of claim 6, wherein the PEG ester is polyoxyl stearate.

8. The abuse deterrent and aversive formulation of claim 6, wherein the poloxamer is poloxamer 124.

9. The abuse-deterrent and aversive formulation of claim 6, wherein the water-soluble anionic polysaccharide is gellan gum.

10. The abuse deterrent and aversive formulation of claim 1, the ratio of poloxamer:polysaccharide:PEG ever is about 40:30:30.
